(19) United States
(12) Patent Application Publication
(10) Pub. No.: US 2026/0297504 A1
(43) Pub. Date: Oct. 1, 2026

(54) MODIFIED MICROORGANISMS AND METHODS OF MAKING AND USING SAME

(71) Applicant: Government of the United States as represented by the Secretary of the Air Force, Wright-Patterson AFB, OH (US)

(72) Inventors: Joseph M. Slocik, Dayton, OH (US); Patrick B. Dennis, Loveland, OH (US); Rajesh R. Naik, Centerville, OH (US); Nancy Kelley-Loughnane, Cincinnati, OH (US)

(21) Appl. No.: 19/576,620

(22) Filed: Mar. 24, 2026

Related U.S. Application Data
(60) Provisional application No. 63/778,577, filed on Mar. 27, 2025.

(51) Int. Cl. C12N 1/20

(52) U.S. Cl. CPC C12N 1/20

(57) ABSTRACT

A process of making modified microorganisms that is less determinantal to cell walls of such microorganisms is disclosed. Such process yields an unexpectedly large improvement in microorganism protein loading, viability, protein activity and environmental tolerance. Thus, modified microorganisms having unexpectedly improved properties and methods of making and using such modified microorganisms is disclosed.

### CROSS-REFERENCE TO RELATED APPLICATIONS

This application claims the benefit of and priority to U.S. Provisional Application Ser. No. 63/778,577, entitled “Modified Microorganisms and Methods of Making and Using Same,” filed on Mar. 27, 2025, the entirety of which is incorporated by reference herein.

### RIGHTS OF THE GOVERNMENT

The invention described herein may be manufactured and used by or for the Government of the United States for all governmental purposes without the payment of any royalty.

### BACKGROUND OF THE INVENTION

Field of the Invention The present invention relates modified microorganisms and methods of making and using same.

### DESCRIPTION OF THE RELATED ART

The removal of water from mixtures of microorganisms in protein liquids is important to achieving intracellular transport of proteins, creating solvent-free “living” materials, controlling recombinant protein expression, and generating temporary new phenotypes. However, the method used to remove water greatly impacts cell viability and intracellular amounts of loaded proteins. Currently, lyophilization is used as a means to modify bacterial cells with protein liquids for example, GFP, IgG antibodies, ferritin proteins, and an RNA polymerase (RNAP).

While lyophilization yields an improvement in this technological area, there is a need in the art for even further enhanced microorganism protein loading, viability, protein activity and environmental tolerance.

### SUMMARY OF THE INVENTION

A process of making modified microorganisms that is less determinantal to cell walls of such microorganisms is presented. Such process yields an unexpectedly large improvement in microorganism protein loading, viability, protein activity and environmental tolerance. Thus, modified microorganisms having unexpectedly improved properties and methods of making and using such modified microorganisms is disclosed.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1A depicts the confocal fluorescence image of E. coli (DH5a with PUC19 plasmid) treated with reconstituted only GFP and air dried;

FIG. 1B depicts the confocal image of E. coli (DH5a with PUC19 plasmid) treated with GFP protein liquid, air dried, reconstituted, washed, and stained with FM4-64 dye;

FIG. 2 depicts the In vivo expression of fluorescent mEos3.2 protein in E. coli DH5a (PST44-mEos3.2 gene) regulated by intercellular transport of T7 RNAP protein liquid;

FIG. 3 is depicts the intercellular transport of E. coli DH5a by Dnase I protein liquid and In vivo degradation of genomic E. coli DNA.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

Applicants recognized that the source of the problem in the lyophilization process was that the low pressures used lysis microorganisms. Thus, Applicants disclose a new process that is less determinantal to cell walls of microorganisms. Such process yields an unexpectedly large improvement in microorganism protein loading, viability, protein activity and environmental tolerance. Thus, Applicants provide herein modified microorganisms having unexpectedly improved properties and methods of making and using such modified microorganisms.

Exemplary Process of Making Intracellular Modification of Microorganisms with Protein Liquids Using a Drying Method

In this exemplary embodiment, a process of making a modified microorganism is disclosed. The process includes drying a protein liquid that has not been frozen. The protein liquid includes a microorganism, a modified protein/anion pair and water. In some embodiments the drying removes at least 79% of the water from the protein liquid. In other embodiments, the drying removes at least 90% of the water from the protein liquid. In still other embodiments, the drying removes at least 90% of the water from the protein liquid. In some of the disclosed embodiments the drying removes at least 98% of the water from the protein liquid. In a particular embodiment, the drying removes from about 79% to about 99.5% of the water from the protein liquid.

In some embodiments, the microorganism included in the protein liquid may be selected from a prokaryote microorganism, a eukaryote microorganism, a yeast and mixtures thereof. The microorganism, in some other embodiments may include gram-negative bacteria, gram-positive bacteria, and mixtures thereof. In still other embodiments, the microorganism includes E. coli mixtures and the modified protein/anion pair may be selected from cationized enzyme/poly(ethylene glycol) 4-nonylphenyl 3-sulfopropyl ether, cationized antibody/poly(ethylene glycol) 4-nonylphenyl 3-sulfopropyl ether, cationized cell regulatory protein/poly(ethylene glycol) 4-nonylphenyl 3-sulfopropyl ether mixtures thereof. In some of these embodiments the cationized enzyme/poly(ethylene glycol) 4-nonylphenyl 3-sulfopropyl ether may be selected from cationized Dnase I/poly(ethylene glycol) 4-nonylphenyl 3-sulfopropyl ether, cationized Cas9/poly(ethylene glycol) 4-nonylphenyl 3-sulfopropyl ether, cationized RNA polymerase/poly(ethylene glycol) 4-nonylphenyl 3-sulfopropyl ether and mixtures thereof. In some other of these embodiments, the cationized RNA polymerase/poly(ethylene glycol) 4-nonylphenyl 3-sulfopropyl ether may be selected from cationized T7 RNA polymerases/poly(ethylene glycol) 4-nonylphenyl 3-sulfopropyl ether, cationized T3 RNA polymerases/poly(ethylene glycol) 4-nonylphenyl 3-sulfopropyl ether and mixtures thereof.

In some embodiments the protein liquid contains, based on total protein liquid weight, from about 1% to about 15% microorganism. In some of these embodiments the protein liquid contains from about 5% to about 15% microorganism, and in some of these embodiments from about 7% to about 12% microorganism, with some preferably from about 8% to about 10% microorganism. In some embodiments the protein liquid contains from about 20% to about 40% modified protein/anion pair. In some of these embodiments, the protein liquid contains from about 25% to about 38% modified protein/anion pair, with some preferably from about 30% to about 36% modified protein/anion pair, and some most preferably from about 33% to about 35% modified protein/anion pair. In these exemplary embodiments, the balance of the protein liquid is water.

In some embodiments, the protein liquid comprises: from about 5% to about 10% of E. coli or C. lytica and from about 20% to about 35% of cationized Cas9 enzyme/poly(ethylene glycol) 4-nonylphenyl 3-sulfopropyl ether anion pair, or from about 5% to about 10% of E. coli or Azospirillium or Bradyrhizobium japonicum and from about 20% to about 35% of Photosystem II complex/poly(ethylene glycol) 4-nonylphenyl 3-sulfopropyl ether anion pair, or from about 5% to about 10% of E. coli and from about 20% to about 35% of Dnase I/poly(ethylene glycol) 4-nonylphenyl 3-sulfopropyl ether anion pair; from about 5% to about 10% of E. coli and from about 20% to about 35% of Proteinase K complex/poly(ethylene glycol) 4-nonylphenyl 3-sulfopropyl ether anion pair. In other embodiments the protein liquid comprises: from about 5% to about 10% of E. coli and from about 20% to about 35% of cationized SulA cytoplasmic protein/poly(ethylene glycol) 4-nonylphenyl 3-sulfopropyl ether anion pair, or from about 5% to about 10% of E. coli and from about 20% to about 35% of cationized β-keratin/poly(ethylene glycol) 4-nonylphenyl 3-sulfopropyl ether anion pair, or from about 5% to about 10% of E. coli and from about 20% to about 35% of cationized cellulase/poly(ethylene glycol) 4-nonylphenyl 3-sulfopropyl ether anion pair. In still other embodiments the protein liquid comprises: from about 5% to about 10% of E. coli and from about 20% to about 35% of cationized lipase/poly(ethylene glycol) 4-nonylphenyl 3-sulfopropyl ether anion pair, or from about 5% to about 10% of E. coli or Sporosarcina pasteurii or Bacillus pasteurii and from about 20% to about 35% of cationized urease/poly(ethylene glycol) 4-nonylphenyl 3-sulfopropyl ether anion pair, or from about 5% to about 10% of E. coli or Azospirillium or Bradyrhizobium japonicum and from about 20% to about 35% of cationized nitrogenase/poly(ethylene glycol) 4-nonylphenyl 3-sulfopropyl ether anion pair.

In some of these disclosed embodiments, drying may include passive drying and/or active drying, preferably the active drying comprises heating the protein liquid to a temperature of 22° C. to about 45° C. under a pressure of about 1 atm. In some of these embodiments the active drying comprises heating the protein liquid to a temperature of 30° C. to about 40° C. under a pressure of about 1 atm. And in yet some of these embodiments, the active drying comprises heating the protein liquid to a temperature of 35° C. to about 37° C. under a pressure of about 1 atm.

In some embodiments, the surface area of the protein liquid is increased prior to or during the drying process by distributing the protein liquid on an object. In some of these embodiments, the object may be a flat surface and/or is non-porous. In yet some of these embodiments, the object may be a metal, a glass and/or a polymer. The glass may comprise borosilicate, the metal may comprise Al, Cu, Ag, and/or Au and the polymer may comprise polypropylene, polyethylene, polycarbonate and/or polyacrylate.

In some embodiments, the drying is conducted for a time of from about 5 minutes to about 1 week. In some of these embodiments, the drying is conducted for a time of from about 10 minutes to about 1 hour. In yet other embodiments, the drying is conducted for a time of from about 15 minutes to about 30 minutes. An exemplary drying time may be conducted for a time of from about minutes 16 to about 20 minutes. Another exemplary drying time may be conducted for a time of at least 1 minute.

Second Process of Making Intracellular Modification of Microorganisms with Protein Liquids Using a Drying Method

In this exemplary embodiment, a process consisting making a modified microorganism is disclosed. The process includes drying a protein liquid that has not been frozen. The protein liquid includes a microorganism, a modified protein/anion pair and water. The drying removes at least 79% of the water from the protein liquid. In some embodiments, the drying removes at least 90% of the water from the protein liquid. In still other embodiments the drying removes at least 90% of the water from the protein liquid. In a particular embodiment, the drying removes at least 98% of the water from the protein liquid. In another particular embodiment, the drying removes from about 79% to about 99.5% of the water from the protein liquid.

In some embodiments, the microorganism is selected from a group consisting of a prokaryote microorganism, a eukaryote microorganism, a yeast and mixtures thereof. In some other embodiments, the microorganism is selected from a group consisting of gram-negative bacteria, gram-positive bacteria and mixtures thereof. In still other embodiments, the microorganism is E. coli mixtures thereof. The modified protein/anion pair may be selected from a group consisting of cationized enzyme/poly(ethylene glycol) 4-nonylphenyl 3-sulfopropyl ether, cationized antibody/poly(ethylene glycol) 4-nonylphenyl 3-sulfopropyl ether, cationized cell regulatory protein/poly(ethylene glycol) 4-nonylphenyl 3-sulfopropyl ether mixtures thereof. In some embodiments, the cationized enzyme/poly(ethylene glycol) 4-nonylphenyl 3-sulfopropyl ether may be selected from a group consisting of cationized Dnase I/poly(ethylene glycol) 4-nonylphenyl 3-sulfopropyl ether, cationized Cas9/poly(ethylene glycol) 4-nonylphenyl 3-sulfopropyl ether, cationized RNA polymerase/poly(ethylene glycol) 4-nonylphenyl 3-sulfopropyl ether and mixtures thereof. In other embodiments, the cationized RNA polymerase/poly(ethylene glycol) 4-nonylphenyl 3-sulfopropyl ether may be selected from a group consisting of cationized T7 RNA polymerases/poly(ethylene glycol) 4-nonylphenyl 3-sulfopropyl ether, cationized T3 RNA polymerases/poly(ethylene glycol) 4-nonylphenyl 3-sulfopropyl ether and mixtures thereof.

In some embodiments, the protein liquid includes, based on total protein liquid weight: from about 1% to about 15% microorganism. In some of these embodiments, the protein liquid includes from about 5% to about 15% microorganism. In some of these embodiments, the protein liquid includes from about 7% to about 12% microorganism. In a particular embodiment, the protein liquid includes from about 8% to about 10% microorganism. In some embodiments, the protein liquid may include from about 20% to about 40% modified protein/anion pair. In some of these embodiments, the protein liquid includes from about 25% to about 38% modified protein/anion pair. And in some of these embodiments, the protein liquid includes from about 30% to about 36% modified protein/anion pair. In a particular embodiment, the protein liquid includes from about 33% to about 35% modified protein/anion pair. The balance of the protein liquid is water.

In some embodiments, the protein liquid may include from about 5% to about 10% of E. coli or C. lytica and from about 20% to about 35% of cationized Cas9 enzyme/poly(ethylene glycol) 4-nonylphenyl 3-sulfopropyl ether anion pair. In some embodiments, the protein liquid includes from about 5% to about 10% of E. coli or Azospirillium or Bradyrhizobium japonicum and from about 20% to about 35% of Photosystem II complex/poly(ethylene glycol) 4-nonylphenyl 3-sulfopropyl ether anion pair. In other embodiments the protein liquid includes from about 5% to about 10% of E. coli and from about 20% to about 35% of Dnase I/poly(ethylene glycol) 4-nonylphenyl 3-sulfopropyl ether anion pair. In still other embodiments the protein liquid includes from about 5% to about 10% of E. coli and from about 20% to about 35% of Proteinase K complex/poly(ethylene glycol) 4-nonylphenyl 3-sulfopropyl ether anion pair. Other include from about 5% to about 10% of E. coli and from about 20% to about 35% of cationized SulA cytoplasmic protein/poly(ethylene glycol) 4-nonylphenyl 3-sulfopropyl ether anion pair or from about 5% to about 10% of E. coli and from about 20% to about 35% of cationized β-keratin/poly(ethylene glycol) 4-nonylphenyl 3-sulfopropyl ether anion pair, or from about 5% to about 10% of E. coli and from about 20% to about 35% of cationized cellulase/poly(ethylene glycol) 4-nonylphenyl 3-sulfopropyl ether anion pair. In some embodiments, the protein liquid includes from about 5% to about 10% of E. coli and from about 20% to about 35% of cationized lipase/poly(ethylene glycol) 4-nonylphenyl 3-sulfopropyl ether anion pair. In other embodiments, the protein liquid includes from about 5% to about 10% of E. coli or Sporosarcina pasteurii or Bacillus pasteurii and from about 20% to about 35% of cationized urease/poly(ethylene glycol) 4-nonylphenyl 3-sulfopropyl ether anion pair. In a particular embodiment the protein liquid may include from about 5% to about 10% of E. coli or Azospirillium or Bradyrhizobium japonicum and from about 20% to about 35% of cationized nitrogenase/poly(ethylene glycol) 4-nonylphenyl 3-sulfopropyl ether anion pair.

In some embodiments, the drying includes passive drying and/or active drying, where preferably the active drying includes heating the protein liquid to a temperature of 22° C. to about 45° C. under a pressure of about 1 atm. In some of these embodiments, the active drying includes heating the protein liquid to a temperature of 30° C. to about 40° C. under a pressure of about 1 atm. In a particular embodiment, the active drying includes heating the protein liquid to a temperature of 35° C. to about 37° C. under a pressure of about 1 atm.

In some embodiments, the surface area of the protein liquid may be increased prior to or during the drying by distributing the protein liquid on an object. The object may be a flat surface and/or is non-porous. In some embodiments, the object may comprise a metal, a glass and/or a polymer. The glass comprises borosilicate, the metal comprises Al, Cu, Ag, and/or Au and the polymer comprises polypropylene, polyethylene, polycarbonate and/or polyacrylate.

In some embodiments, the drying is conducted for a time of from about 5 minutes to about 1 week. In some of these embodiments, the drying is conducted for a time of from about 10 minutes to about 1 hour. In some of these embodiments, the drying is conducted for a time of from about 15 minutes to about 30 minutes. In a particular embodiment, the drying is conducted for a time of from about minutes 16 to about 20 minutes. In another particular embodiment, the drying is conducted for a time of at least 1 minute.

### Exemplary Modified Microorganism

An exemplary modified microorganism is presented having a protein loading index of 2% to 80%. In some embodiments, the loading index may be from about 20% to about 80%. In some of these embodiments, the loading index may be from about 50% to about 80%. In a particular embodiment, the loading index may be from about 70% to about 80%. In some embodiments the microorganism includes at least one of: a microorganism viability index of 0.05 to 0.90. In some of these embodiments, the viability index may be from about 0.2 to about 0.9. In some of these embodiments the viability index may be from about 0.5 to about 0.9. In a particular embodiment the viability index may be from about 0.8 to about 0.9. In some embodiments, the microorganism includes a protein activity index of 1 to 10. In some of these embodiments, the protein activity index may be from about 2 to about 10. In some of these embodiments, the protein activity index may be from about 5 to about 10. In a particular embodiment, the protein activity index may be from about 8 to about 10. And, the microorganism includes an environmental tolerance index of 0.02 to 0.8. In some of these embodiments, the environmental tolerance index may be from about 0.2 to about 0.8. In some of these embodiments, the environmental tolerance index may be from about 0.5 to about 0.8, In a particular embodiment, the environmental tolerance index may be from about 0.7 to about 0.8. Preferably said modified microorganism has the protein loading index and at least two of the remaining indices, more preferably the modified microorganism has protein loading index and all three of the remaining indices.

In some embodiments, the modified microorganism of the previous paragraph may be selected from the group consisting of a modified prokaryote microorganism, a modified eukaryote microorganism, and/or a modified, preferably said modified microorganism is a modified gram-negative bacteria and/or a modified gram-positive bacteria and mixtures thereof, more preferably said microorganism is a modified E. coli.

### Test Methods

Protein Loading Index: A high protein loading is important because it increases the probability of microorganisms becoming modified by protein liquids, increases molecular crowding inside microorganisms, and maximizes protein activity for controlling internal cellular processes. As an illustrated exemplary embodiment, the protein loading of a modified microorganism is provided via a protein loading index that is determined as follows: Microorganisms were treated and air dried in protein liquids comprised of fluorescent proteins, reconstituted in 100 μL of doubly deionized water, centrifuged at 8000 rpm for 5 minutes to pellet cells and remove supernatant, and redispersed in fresh doubly deionized water. Confocal fluorescence microscopy was performed using a Zeiss LSM700 confocal laser scanning microscope with 488 nm excitation argon laser. Samples of microorganisms treated with protein liquids were prepared for imaging by pipetting 10 μL of treated cells onto a borosilicate glass microscope slide and air dried. Dried bacteria samples were fixed on glass slides by flame annealing and then fluorescently stained by placing 10 μL of a FM4-64 stock solution in water on dried cell spot. Immediately, a glass coverslip was placed on top of droplet and sealed with clear nail polish to prevent drying. A set of 5 confocal fluorescence images were collected over different regions of a microscope slide that contained 20-100 rod-shaped, spherical, or filament shapes of microorganisms for all untreated and treated microorganisms using the same excitation, emission, and exposure parameters. Automated image analysis was performed on image set using MicrobeJ to segment bacteria and produce intensity histograms. Each intensity histogram was used to generate the total GFP intensity/population of microorganisms. This value was divided by the total GFP intensity for untreated microorganisms to yield a protein loading index based on a percentage scale.

Microorganism Viability Index: A high microorganism viability is important because it ensures a high fraction of metabolically active cells in the total population. In the illustrative embodiment, the microorganism viability of a modified microorganism is provided via a microorganism viability in microorganism viability index that is determined as follows: Microorganisms dried with/without protein liquid were reconstituted and plated on LB agar plates in the presence of ampicillin using serial dilutions (100, 10-1, 10-2, 10-3, 10-4, 10-5, 10-6). LB agar plates were grown at 37° C. for 18 hours. The number of colonies that grew on LB plate after 18 hours were counted at the lowest dilution and divided by volume of cell spot (5 μL) to yield the number of colony forming units/mL (CFU/mL). A viability index was determined by dividing the CFU/mL value from protein liquid treated and air-dried microorganisms by the CFU/mL of untreated microorganisms before modifications. Using this scale, an index value of 0 represents complete microorganism death while a value of 1 signifies all microorganisms are metabolically active and alive.

Protein Activity Index: A protein activity is important because it determines the effectiveness of protein liquids at regulating/controlling internal cellular processes. In this illustrative embodiment, the protein activity of a modified microorganism is provided via a protein activity index that is determined as follows: Microorganisms deficient in T7 RNA polymerase and transformed with a gene expressing mEos3.2 fluorescent protein were treated and air dried with protein liquids comprising a T7 RNA polymerase. Upon drying, microorganisms treated with/without T7 RNA polymerase were reconstituted in LB/ampicillin, transferred to a black 96 well plate with clear bottom, and measured for protein production by monitoring protein fluorescence over time on a BioTek Synergy Neo2 multi-mode well plate reader. For fluorescence measurements, the excitation was set to 500 nm and emission at 515 nm, temperature was controlled at 37° C., and a measurement was collected at 30 minute intervals for 8 hours. The final fluorescence value representing the total protein expressed as a result of T7 RNA polymerase activity was divided by the starting fluorescence value at time 0. This value was evaluated on a scale from 0 to 10; whereby 0 represents no protein activity, 4 represents a relatively high protein activity, and 10 represents extremely high protein activity.

Environmental Tolerance Index: A high environmental tolerance is important because it protects microorganisms against cell death upon exposure to extreme environmental conditions. In this illustrative embodiment, the environmental tolerance of a modified microorganism is provided via an environmental tolerance index that is determined as follows: Microorganisms treated with/without protein liquids were air dried on a polypropylene petri plate, covered with matching polystyrene lid, and heated at 95° C. for 20 minutes. After heating, the samples were cooled to room temperature for approximately 10 minutes and plated on LB agar plates in the presence of ampicillin using serial dilutions (10⁰, 10⁻¹, 10⁻², 10⁻³, 10⁻⁴, 10⁻⁵, 10⁻⁶). LB agar plates were grown at 37° C. for 18 hours and counted for number of colonies. An environmental tolerance index was calculated by dividing the CFU/mL value from protein liquid treated and air-dried microorganisms that were heated at 95° C. for 20 minutes by the CFU/mL of untreated microorganisms before modifications at room temperature and evaluated on a scale of 0 to 1; whereby, 0 represents complete microorganism death at specified temperature, while 0.5 represents 50% survival of the microorganisms and 1 represents survival of all microorganisms.

### Examples

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

The following Tables 1-5 below are referenced in the examples.

TABLE 1
Fluorescence intensities of expressed mEos3.2 protein over time
for E. coli only and E. coli treated with T7 RNAP protein
liquid, native T7 RNAP, and ferritin protein liquid measured
on a microplate reader. (Exc. 500 nm/Em. 515 nm).

| | 0 hr | 1 hr | 2 hrs | 4 hrs |
|---|---|---|---|---|
| Cells only | 520 | 514 | 513 | 519 |
| Cells + Native T7 RNAP | 595 | 593 | 578 | 587 |
| Cells + T7 RNAP protein liquid | 548 | 577 | 958 | 1331 |
| Cells + Ferritin protein liquid | 567 | 554 | 566 | 616 |

TABLE 2
Fluorescence intensities of expressed mEos3.2 protein
over time for E. coli only and E. coli treated
with 1 μg, 10 μg, and 20 μg of T7 RNAP protein liquid.

| | 0 hr | 2 hrs | 4 hrs | 6 hrs | 8 hrs |
|---|---|---|---|---|---|
| Cells only | 528 | 509 | 512 | 515 | 521 |
| Cells + 1 μg T7 RNAP | 537 | 654 | 567 | 549 | 544 |
| protein liquid | | | | | |
| Cells + 10 μg T7 RNAP | 539 | 862 | 948 | 1011 | 1064 |
| protein liquid | | | | | |
| Cells + 20 μg T7 RNAP | 547 | 1166 | 1327 | 1417 | 1499 |
| protein liquid | | | | | |

TABLE 3
Cell viability of air dried E. coli (DH5α with PUC19)
treated with controls or PSII protein liquid at room temperature
and after exposure to 95° C. heating for 20 minutes.

| | Cell Viability | Cell Viability @ |
|---|---|---|
| | @RT (CFU/mL) | 95° C. (CFU/mL) |
| Cells only | 3.5 × 10⁵ | 0 |
| Cells + Anion only | 2.1 × 10⁴ | 0 |
| Cells + BmimCl | 0 | 0 |
| Cells + PSII protein liquid | 2.7 × 10⁵ | 1.3 × 10⁴ |

TABLE 4
Cell viability of air dried E. coli, C. lytica, and
B. subtilis treated with PSII protein liquid at room
temperature and after exposure to 95° C. for 20 minutes.

| | Cell Viability | Cell Viability @ |
|---|---|---|
| | @ RT (CFU/mL) | 95° C. (CFU/mL) |
| E. coli + PSII | 2.7 × 10⁵ | 1.0 × 10⁵ |
| protein liquid | | |
| C. lytica + PSII | 1.4 × 10⁶ | 8.3 × 10⁵ |
| protein liquid | | |
| B. subtilis + PSII | 5.3 × 10⁵ | 9.0 × 10² |
| protein liquid | | |

TABLE 5
Cell viability plot of cells only, cells treated with native Dnase I,
cells treated with a heat-inactivated Dnase I protein liquid, and cells
treated with 0.1 mg and 0.3 mg of active Dnase I protein liquid.

| | Heat | Amount | Cell Viability |
|---|---|---|---|
| | inactivated | Dnase I | (CFU/mL) |
| Cells only | N/A | 0 | 1.8 × 109 |
| Cells + Native Dnase I | No | 0.1 mg | 1.5 × 108 |
| Cells + Dnase I Protein liquid | Yes | 0.1 mg | 2.2 × 108 |
| Cells + Dnase I Protein liquid | No | 0.1 mg | 4.7 × 106 |
| Cells + Dnase I Protein liquid | No | 0.3 mg | 1.7 × 105 |

### Example 1: Intracellular Delivery of GFP Protein Liquid in E. coli

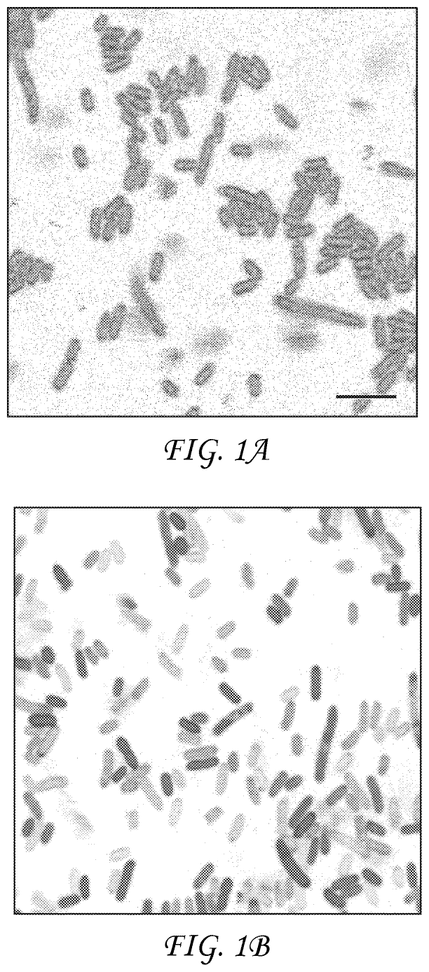

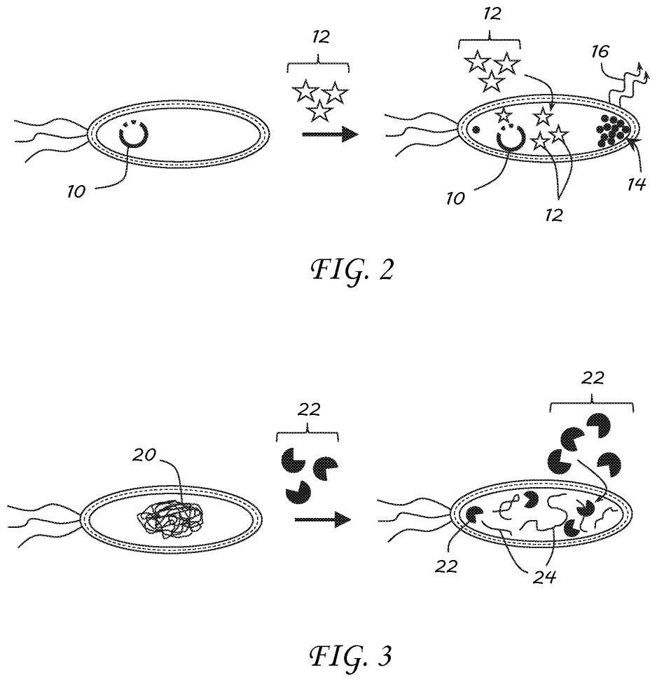

E. coli (DH5a strain) transformed with PUC19 plasmid (~2×10⁹ stationary phase cells) was added to 20 μL of a reconstituted aqueous protein liquid solution of green fluorescent protein (GFP) at 75 mg/mL to ensure a homogeneous suspension of cells, spread onto a sterile non-treated 35×20 mm petri plate to increase surface area and evaporation rate, and air dried at room temperature for 15-30 minutes. After drying, E. coli dried in GFP ionic liquid was reconstituted in 100 μL of doubly deionized water, centrifuged at 8000 rpm for 5 minutes to pellet cells and remove supernatant, and redispersed in fresh doubly deionized water. Air dried and washed E. coli cells in GFP ionic liquid were stained with a red fluorescent membrane stain (FM4-64) and imaged by confocal microscopy for GFP fluorescence. Confocal images, such as those in FIGS. 1A and 1B, showed cross-sections of bacteria containing high amounts of internalized GFP protein liquid. The modified microorganism of the present example was tested according to the test methods found in the test methods section of this specification and found to have an 80% protein loading index.

### Example 2: Intracellular Delivery of T7 TNAP Protein Liquid in E. coli and In Vivo Expression of mEos3.2 Fluorescent Protein

E. coli (DH5a strain) transformed with a PST44-mEos3.2 plasmid (~2×10⁹ stationary phase cells) 10 was added to 20 μL of a reconstituted aqueous protein ionic liquid solution of T7 RNA polymerase (T7 RNAP) 12 at 75 mg/mL to ensure a homogeneous suspension of cells, spread onto a sterile non-treated 35×20 mm petri plate to increase surface area and evaporation rate, and air dried at room temperature for 15-30 minutes as diagrammatically illustrated in FIG. 2. After drying, E. coli in T7 RNAP ionic liquid was reconstituted in 200 μL of LB media with ampicillin and measured for mEos3.2 protein 14 expression by fluorescence 16 using a microplate reader over time at 37° C. In parallel, transformed cells were similarly treated with unmodified T7 RNAP and a ferritin ionic liquid. As a result, T7 RNAP ionic liquid treated cells showed a sharp increase in fluorescence at 515 nm due to expression of fluorescent mEos3.2 protein after a short lag time; while cells treated with unmodified T7 RNAP and ferritin ionic liquid showed no increase in fluorescence. At the same time, we showed protein expression was strictly regulated by changing the amount of T7 RNAP ionic liquid added to E. coli and directly proportional to the fluorescence output. Table 1 above provides the fluorescence intensities of expressed mEos3.2 protein over time for E. coli only and E. coli treated with T7 RNAP protein liquid, native T7 RNAP, and ferritin protein liquid measured on a microplate reader. (Exc. 500 nm/Em. 515 nm) for this example. Table 2 above provides fluorescence intensities of expressed mEos3.2 protein over time for E. coli only and E. coli treated with 1 μg, 10 μg, and 20 μg of T7 RNAP protein liquid for this example. The modified microorganism of the present example was tested according to the test methods found in the teat methods section of this specification and found to have a 2.4 protein activity index.

### Example 3: Intracellular Delivery of Photosystem II Protein Liquid in E. coli and Thermal Stabilization

E. coli (DH5a strain) transformed with a PUC19 plasmid (~2× 109 stationary phase cells) was added to 20 μL of a reconstituted aqueous protein ionic liquid solution of photosystem II extracted from spinach (PSII) at 75 mg/mL to ensure a homogeneous suspension of cells, spread onto a sterile non-treated 35×20 mm petri plate to increase surface area and evaporation rate, and air dried at room temperature for 15-30 minutes. Air dried samples containing cells dispersed in PSII protein liquid or controls were kept at room temperature or heated at 95° C. for 20 minutes, reconstituted in 0.01 M Tris HCl, and plated by serial dilutions for colony counting. Consequently, the set of controls revealed 100% cell death after 95° C. heat treatment. E. coli suspended in reconstituted membrane-free PSII protein ionic liquid and air dried showed a high survival number of viable cells after heating at 95° C. Table 3 above provides the cell viability of air dried E. coli (DH5a with PUC19) treated with controls or PSII protein liquid at room temperature and after exposure to 95° C. heating for 20 minutes and Table 4 provides the cell viability of air dried E. coli, C. lytica, and B.subtilis treated with PSII protein liquid at room temperature and after exposure to 95° C. for 20 minutes. The modified microorganism of the present example was tested according to the test methods found in the teat methods section of this specification and found to have a 0.05 environmental tolerance index at 95° C.

### Example 4: Intracellular Delivery of Dnase I Protein Liquid in E. coli and In Vivo DNA Degradation

E. coli (DH5a strain) transformed with a PUC19 plasmid (~2×10⁹ stationary phase cells) 20 was added to 20 μL of a reconstituted aqueous protein ionic liquid solution of Dnase I at 75 mg/mL to ensure a homogeneous suspension of cells, spread onto a sterile non-treated 35×20 mm petri plate to increase surface area and evaporation rate, and air dried at room temperature for 15-30 minutes as diagrammatically illustrated in FIG. 2. Air dried samples containing cells dispersed in Dnase I protein liquid 22 or native unmodified Dnase I were reconstituted in 0.01 M Tris HCl and plated by serial dilutions for colony counting and for the in vivo degradation of genomic DNA (~5.5 Mb) in E. coli 24. In parallel, cells were also treated cells with a heat inactivated form of [C-Dnase][S] at the same concentration in order to account for cell viability losses due to drying on petri plate and exposure to protein liquid. Upon plating, we observed a ~1 log loss in CFUs for both cells treated with unmodified Dnase I and heat inactivated [C-DNase][S] protein liquid. However, cells treated and air dried with [C-Dnase][S] showed a ~3 log and ~4 log decrease in CFUs depending on amount of added [C-Dnase][S]. In this case, protein liquid treated E. coli cells are unable to replicate due to [C-Dnase][S] induced DNA cleavage and did not form colonies. Quantitatively, cell death percentages of 98% and 99.93% were obtained for the total population of cells using 0.1 mg and 0.3 mg of Dnase I protein liquid, respectively. Table 5 provides the cell viability plot of cells only, cells treated with native Dnase I, cells treated with a heat-inactivated Dnase I protein liquid, and cells treated with 0.1 mg and 0.3 mg of active Dnase I protein liquid. The modified microorganism of the present example was tested according to the test methods found in the teat methods section of this specification and found to have a 0.00009 microorganism viability index.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A process of making a modified microorganism comprising drying a protein liquid that has not been frozen, said protein liquid comprising a microorganism, a modified protein/anion pair and water, said drying removing at least 79% of said water from said protein liquid, preferably said drying removing at least 90% of said water from said protein liquid, more preferably said drying removing at least 90% of said water from said protein liquid, most said drying removing at least 98% of said water from said protein liquid, in one aspect said drying removes from about 79% to about 99.5% of said water from said protein liquid.

2. The process of claim 1 wherein:
said microorganism is selected from the group consisting of a prokaryote microorganism, a eukaryote microorganism, a yeast and mixtures thereof, preferably said microorganism is selected from the group consisting of gram-negative bacteria gram-positive bacteria and mixtures thereof, more preferably said microorganism is E. coli mixtures thereof; and
said modified protein/anion pair is selected from the group consisting of cationized enzyme/poly(ethylene glycol) 4-nonylphenyl 3-sulfopropyl ether, cationized antibody/poly(ethylene glycol) 4-nonylphenyl 3-sulfopropyl ether, cationized cell regulatory protein/poly(ethylene glycol) 4-nonylphenyl 3-sulfopropyl ether mixtures thereof, preferably said cationized enzyme/poly(ethylene glycol) 4-nonylphenyl 3-sulfopropyl ether is selected from the group consisting of cationized Dnase I/poly(ethylene glycol) 4-nonylphenyl 3-sulfopropyl ether, cationized Cas9/poly(ethylene glycol) 4-nonylphenyl 3-sulfopropyl ether, cationized RNA polymerase/poly(ethylene glycol) 4-nonylphenyl 3-sulfopropyl ether and mixtures thereof, preferably said cationized RNA polymerase/poly(ethylene glycol) 4-nonylphenyl 3-sulfopropyl ether is selected from the group consisting of cationized T7 RNA polymerases/poly(ethylene glycol) 4-nonylphenyl 3-sulfopropyl ether, cationized T3 RNA polymerases/poly(ethylene glycol) 4-nonylphenyl 3-sulfopropyl ether and mixtures thereof.

3. The process of claim 2 wherein said protein liquid comprises, based on total protein liquid weight:
from about 1% to about 15% microorganism, preferably from about 5% to about 15% microorganism, more preferably from about 7% to about 12% microorganism, most preferably from about 8% to about 10% microorganism;
from about 20% to about 40% modified protein/anion pair; preferably from about 25% to about 38% modified protein/anion pair, more preferably from about 30% to about 36% modified protein/anion pair, most preferably from about 33% to about 35% modified protein/anion pair; and
the balance of said protein liquid being said water.

4. The process of claim 3 wherein said protein liquid comprises:
from about 5% to about 10% of E. coli or C. lytica and from about 20% to about 35% of cationized Cas9 enzyme/poly(ethylene glycol) 4-nonylphenyl 3-sulfopropyl ether anion pair;
from about 5% to about 10% of E. coli or Azospirillium or Bradyrhizobium japonicum and from about 20% to about 35% of Photosystem II complex/poly(ethylene glycol) 4-nonylphenyl 3-sulfopropyl ether anion pair;
from about 5% to about 10% of E. coli and from about 20% to about 35% of Dnase I/poly(ethylene glycol) 4-nonylphenyl 3-sulfopropyl ether anion pair;
from about 5% to about 10% of E. coli and from about 20% to about 35% of Proteinase K complex/poly(ethylene glycol) 4-nonylphenyl 3-sulfopropyl ether anion pair;
from about 5% to about 10% of E. coli and from about 20% to about 35% of cationized SulA cytoplasmic protein/poly(ethylene glycol) 4-nonylphenyl 3-sulfopropyl ether anion pair;
from about 5% to about 10% of E. coli and from about 20% to about 35% of cationized β-keratin/poly(ethylene glycol) 4-nonylphenyl 3-sulfopropyl ether anion pair;
from about 5% to about 10% of E. coli and from about 20% to about 35% of cationized cellulase/poly(ethylene glycol) 4-nonylphenyl 3-sulfopropyl ether anion pair;
from about 5% to about 10% of E. coli and from about 20% to about 35% of cationized lipase/poly(ethylene glycol) 4-nonylphenyl 3-sulfopropyl ether anion pair;
from about 5% to about 10% of E. coli or Sporosarcina pasteurii or Bacillus pasteurii and from about 20% to about 35% of cationized urease/poly(ethylene glycol) 4-nonylphenyl 3-sulfopropyl ether anion pair;
from about 5% to about 10% of E. coli or Azospirillium or Bradyrhizobium japonicum and from about 20% to about 35% of cationized nitrogenase/poly(ethylene glycol) 4-nonylphenyl 3-sulfopropyl ether anion pair.

5. The process of claim 4 wherein said drying comprises passive drying and/or active drying, preferably said active drying comprises heating said protein liquid to a temperature of 22° C. to about 45° C. under a pressure of about 1 atm, more preferably said active drying comprises heating said protein liquid to a temperature of 30° C. to about 40° C. under a pressure of about 1 atm, most preferably said active drying comprises heating said protein liquid to a temperature of 35° C. to about 37° C. under a pressure of about 1 atm.

6. The process of claim 5 wherein the surface area of said protein liquid is increased prior to or during said drying by distributing said protein liquid on an object, preferably said object is a flat surface and/or is non-porous, more preferably said object comprises a metal, a glass and/or a polymer, most preferably said glass comprises borosilicate, said metal comprises Al, Cu, Ag, and/or Au and said polymer comprises polypropylene, polyethylene, polycarbonate and/or polyacrylate.

7. The process of claim 6 wherein said drying is conducted for a time of from about 5 minutes to about 1 week, preferably said drying is conducted for a time of from about 10 minutes to about 1 hour, more preferably said drying is conducted for a time of from about 15 minutes to about 30 minutes, most preferably said drying is conducted for a time of from about minutes 16 to about 20 minutes. In one aspect, said drying is conducted for a time of at least 1 minute.

8. A process consisting making a modified microorganism comprising drying a protein liquid that has not been frozen, said protein liquid comprising a microorganism, a modified protein/anion pair and water, said drying removing at least 79% of said water from said protein liquid, preferably said drying removing at least 90% of said water from said protein liquid, more preferably said drying removing at least 90% of said water from said protein liquid, most said drying removing at least 98% of said water from said protein liquid, in one aspect said drying removes from about 79% to about 99.5% of said water from said protein liquid.

9. The process of claim 8 wherein said microorganism is selected from the group consisting of a prokaryote microorganism, a eukaryote microorganism, a yeast and mixtures thereof, preferably said microorganism is selected from the group consisting of gram-negative bacteria gram-positive bacteria and mixtures thereof, more preferably said microorganism is E. coli mixtures thereof; and the modified protein/anion pair is selected from the group consisting of cationized enzyme/poly(ethylene glycol) 4-nonylphenyl 3-sulfopropyl ether, cationized antibody/poly(ethylene glycol) 4-nonylphenyl 3-sulfopropyl ether, cationized cell regulatory protein/poly(ethylene glycol) 4-nonylphenyl 3-sulfopropyl ether mixtures thereof, preferably said cationized enzyme/poly(ethylene glycol) 4-nonylphenyl 3-sulfopropyl ether is selected from the group consisting of cationized Dnase I/poly(ethylene glycol) 4-nonylphenyl 3-sulfopropyl ether, cationized Cas9/poly(ethylene glycol) 4-nonylphenyl 3-sulfopropyl ether, cationized RNA polymerase/poly(ethylene glycol) 4-nonylphenyl 3-sulfopropyl ether and mixtures thereof, preferably said cationized RNA polymerase/poly(ethylene glycol) 4-nonylphenyl 3-sulfopropyl ether is selected from the group consisting of cationized T7 RNA polymerases/poly(ethylene glycol) 4-nonylphenyl 3-sulfopropyl ether, cationized T3 RNA polymerases/poly(ethylene glycol) 4-nonylphenyl 3-sulfopropyl ether and mixtures thereof.

10. The process of claim 9 wherein said protein liquid comprises, based on total protein liquid weight: from about 1% to about 15% microorganism, preferably from about 5% to about 15% microorganism, more preferably from about 7% to about 12% microorganism, most preferably from about 8% to about 10% microorganism; from about 20% to about 40% modified protein/anion pair; preferably from about 25% to about 38% modified protein/anion pair, more preferably from about 30% to about 36% modified protein/anion pair, most preferably from about 33% to about 35% modified protein/anion pair; and the balance of said protein liquid being said water.

11. The process of claim 10 wherein said protein liquid comprises: from about 5% to about 10% of E. coli or C. lytica and from about 20% to about 35% of cationized Cas9 enzyme/poly(ethylene glycol) 4-nonylphenyl 3-sulfopropyl ether anion pair; from about 5% to about 10% of E. coli or Azospirillium or Bradyrhizobium japonicum and from about 20% to about 35% of Photosystem II complex/poly(ethylene glycol) 4-nonylphenyl 3-sulfopropyl ether anion pair; from about 5% to about 10% of E. coli and from about 20% to about 35% of Dnase I/poly(ethylene glycol) 4-nonylphenyl 3-sulfopropyl ether anion pair; from about 5% to about 10% of E. coli and from about 20% to about 35% of Proteinase K complex/poly(ethylene glycol) 4-nonylphenyl 3-sulfopropyl ether anion pair; from about 5% to about 10% of E. coli and from about 20% to about 35% of cationized SulA cytoplasmic protein/poly(ethylene glycol) 4-nonylphenyl 3-sulfopropyl ether anion pair; from about 5% to about 10% of E. coli and from about 20% to about 35% of cationized β-keratin/poly(ethylene glycol) 4-nonylphenyl 3-sulfopropyl ether anion pair; from about 5% to about 10% of E. coli and from about 20% to about 35% of cationized cellulase/poly(ethylene glycol) 4-nonylphenyl 3-sulfopropyl ether anion pair; from about 5% to about 10% of E. coli and from about 20% to about 35% of cationized lipase/poly(ethylene glycol) 4-nonylphenyl 3-sulfopropyl ether anion pair; from about 5% to about 10% of E. coli or Sporosarcina pasteurii or Bacillus pasteurii and from about 20% to about 35% of cationized urease/poly(ethylene glycol) 4-nonylphenyl 3-sulfopropyl ether anion pair; from about 5% to about 10% of E. coli or Azospirillium or Bradyrhizobium japonicum and from about 20% to about 35% of cationized nitrogenase/poly(ethylene glycol) 4-nonylphenyl 3-sulfopropyl ether anion pair.

12. The process of claim 11 wherein said drying comprises passive drying and/or active drying, preferably said active drying comprises heating said protein liquid to a temperature of 22° C. to about 45° C. under a pressure of about 1 atm, more preferably said active drying comprises heating said protein liquid to a temperature of 30° C. to about 40° C. under a pressure of about 1 atm, most preferably said active drying comprises heating said protein liquid to a temperature of 35° C. to about 37° C. under a pressure of about 1 atm.

13. The process of claim 12 wherein the surface area of said protein liquid is increased prior to or during said drying by distributing said protein liquid on an object, preferably said object is a flat surface and/or is non-porous, more preferably said object comprises a metal, a glass and/or a polymer, most preferably said glass comprises borosilicate, said metal comprises Al, Cu, Ag, and/or Au and said polymer comprises polypropylene, polyethylene, polycarbonate and/or polyacrylate.

14. The process of claim 13 wherein said drying is conducted for a time of from about 5 minutes to about 1 week, preferably said drying is conducted for a time of from about 10 minutes to about 1 hour, more preferably said drying is conducted for a time of from about 15 minutes to about 30 minutes, most preferably said drying is conducted for a time of from about minutes 16 to about 20 minutes. In one aspect, said drying is conducted for a time of at least 1 minute.

15. A modified microorganism having a protein loading index of 2% to 80%; preferably from about 20% to about 80%, more preferably from about 50% to about 80%, most preferably from about 70% to about 80%; and at least one of:
a microorganism viability index of 0.05 to 0.90, preferably from about 0.2 to about 0.9, more preferably from about 0.5 to about 0.9, most preferably from about 0.8 to about 0.9;
a protein activity index of 1 to 10, preferably from about 2 to about 10, more preferably from about 5 to about 10, most preferably from about 8 to about 10; and
an environmental tolerance index of 0.02 to 0.8, preferably from about 0.2 to about 0.8, more preferably from about 0.5 to about 0.8, most preferably from about 0.7 to about 0.8. Preferably said modified microorganism has said protein loading index and at least two of said remaining indices, more preferably said modified microorganism has protein loading index and all three of said remaining indices.

16. The modified microorganism of claim 15, wherein said modified microorganism is selected from the group consisting of a modified prokaryote microorganism, a modified eukaryote microorganism, and/or a modified, preferably said modified microorganism is a modified gram-negative bacteria and/or a modified gram-positive bacteria and mixtures thereof, more preferably said microorganism is a modified E. coli.
